(19) United States
(12) Patent Application Publication
(10) Pub. No.: US 2010/0129464 A1
(43) Pub. Date: May 27, 2010

(54) Manufacturing method and apparatus for producing substances that include negative hydrogen ion

(76) Inventor: Isao Suzuki, Santa Ana, CA (US)

Correspondence Address: MURAMATSU & ASSOCIATES;Suite 310, 114 Pacifica, Irvine, CA, 92618, US

(21) Appl. No.: 12/313,535

(22) Filed: Nov. 21, 2008

(51) Int. Cl. A61K 33/00; A61P 43/00; A23L 3/34

(52) U.S. Cl. 424/600; 426/321

(57) ABSTRACT

A manufacturing method and apparatus produces various medium such as mineral substance, vitamin substance, or ceramic that include negative hydrogen icons for use by human beings and animals. In the manufacturing method, an optional mineral substance or vitamin substance as the medium in granular or powdered form is supplied to the inside of the container and filled with liquid hydrogen or hydrogen, gas. Then after leaving them in the container for a predetermined time, the medium is removed to be air dried, thereby producing the medium which is combined and included with negative hydrogen ion. Alternatively, high pressure, heat, or high voltage may be applied to the medium to contain the negative hydrogen ion in the medium.

### FIELD OF THE INVENTION

This invention relates to a manufacturing method and apparatus for producing various substances that include negative hydrogen icons, and more particularly, to a method and apparatus for producing mineral substance, vitamin substance, or ceramic that include negative hydrogen ions for use by human beings and animals.

### BACKGROUND OF THE INVENTION

Improvement of health is a serious and important issue for human life today. In recent years, it has been found that negative ion excels in anti-oxidation, therefore, products such as air purifiers, air conditioners, and hair dryers have been widely manufactured and used. Further, water purifiers for electrically separating tap water into acidic ion water and alkaline ion water are manufactured and marketed as well.

In the environment that surround people all over the country, there is a large influence of active enzymes such as free-radical oxygen that are introduced or otherwise supplied to the human body. The radical oxygen within the human body as well as those that enter the body harm the cells and genes, which are the reasons for healthy human body to become sick. Further, the advancement of human body's oxidation accelerates the aging process. In addition to the human body, the oxidation in food will lead to damage or decomposition of the food.

Therefore, by incorporating hydrogen into the human body and food, the radical oxygen can be neutralized, and by eliminating the damage caused by the active enzymes, cell activation and metabolism can be enhanced to improve the health of the human body. Further, by incorporating the hydrogen in the food, it is possible to prevent mold, extend life time of the food, and reduce the amount of preservatives that are used for the food. As for tap water, chlorine and organic matter react to form the carcinogenic trihalomethane, and the chlorine harms the human cells as well. The negative hydrogen ion (H−) can eliminate such problems and promote safety of the human body.

As noted above, the negative hydrogen ion is useful to improve the living environment such as purifying the drinking air, etc. In addition, the alkaline reduced water produced by an electronic device in the market can increase alkaline substance. However, the hydrogen in the water produced by such an electronic device is released in a short period of time from the water. Thus, at present, the hydrogen itself cannot find any connection to ecology, such as food, drinking water, supplements, etc.

### SUMMARY OF THE INVENTION

It is, therefore, an object of the present invention to provide a method and apparatus for producing various substances that include negative hydrogen icons in a manner that the negative hydrogen icons stay in the substances.

It is another object of the present invention to provide a method and apparatus for producing various substances or medium such as mineral substance, vitamin substance, or ceramic that include negative hydrogen ions in a manner that the negative hydrogen icons stay in the substances for use by human beings and animals.

In the present invention, an optional mineral substance or vitamin substance as the medium in granular or powdered form is supplied to the inside of the container which is filled with liquid hydrogen or hydrogen gas. Then after leaving them in the container for a predetermined time length such as about 30 minutes to 3 hours, the medium is removed to be air dried, thereby producing the medium which is combined and included with negative hydrogen ion.

Alternatively, after leaving the medium and the liquid hydrogen for the predetermined time (about 30 minutes to 3 hours), the liquid hydrogen is removed. Then, the medium is heated at a predetermined temperature such as about 40-80° C., thereby producing the medium which is combined and included with negative hydrogen ion.

Alternatively, after leaving the medium and the liquid hydrogen for the predetermined time (about 30 minutes to 3 hours), the liquid hydrogen is removed. Then, the medium is pressurized at a predetermined pressure level such as about 5-1000 atm) to produce a plasma state so that hydrogen atom in the medium is converted to the negative hydrogen ion (H−), thereby producing the medium which is combined and included with negative hydrogen ion.

Alternatively, after leaving the medium and the liquid hydrogen for the predetermined time (about 30 minutes to 3 hours), the liquid hydrogen or hydrogen gas is removed. Then, the medium is heated at a predetermined temperature such as about 500-1,000° C. for a predetermined time length such as 1-4 hours to produce a plasma state so that hydrogen atom in the medium is converted to the negative hydrogen ion (H−), thereby producing the medium which is combined and included with negative hydrogen ion.

Alternatively, after leaving the medium and the liquid hydrogen for the predetermined time (about 30 minutes to 3 hours), the liquid hydrogen or hydrogen gas is removed. The air inside of the container is also removed to create an almost absolute vacuum state in the container. Then, the medium is exposed to a high voltage such as about 10,000-100,000 volts for a predetermined time length such as 10-60 minutes to produce a plasma state so that hydrogen atom in the medium is excited and converted to the negative hydrogen ion (H−), thereby producing the medium which is combined and included with negative hydrogen ion.

Alternatively, after leaving the medium and the liquid hydrogen for the predetermined time (about 30 minutes to 3 hours), the liquid hydrogen is removed. The hydrogen gas remains in the container. Then, the medium is exposed to a high voltage such as about 10,000-100,000 volts for a predetermined time length such as 10-60 minutes to produce a plasma state so that hydrogen atom in the medium is excited and converted to the negative hydrogen ion (H−), thereby producing the medium which is combined and included with negative hydrogen ion.

In the manufacturing method, the mineral substance or vitamin substance including the negative hydrogen ion (H−) and hydrogen ion through the treatment in the manufacturing method noted above is formed into capsules, tablets, powder, granules, in tubes or gel for medical purposes and supplemental purposes to be taken by humans, animals, and plants.

In the further aspect, powdered mineral inclusion substance and iron, calcium, magnesium, silicon and organic substance are mixed at a predetermined mixture ratio. The mixed substances are sintered to form ceramic in a porous manner with a desired size. The ceramic is supplied to the inside of the container and filled with liquid hydrogen or hydrogen gas. Then after leaving them in the container for a predetermined time such as about 30 minutes to 3 hours, the liquid hydrogen or hydrogen gas is removed. The air inside of the container 30 is also removed to create an absolute or substantial vacuum state in the container. Then, the ceramic is exposed to a high voltage such as about 10,000-100,000 volts for a predetermined time length such as 10-60 minutes to produce a plasma state so that hydrogen atom in the ceramic is excited and converted to the negative hydrogen ion (H−), thereby producing the ceramic which is combined and included with negative hydrogen ion. Then, the ceramic is removed from the container and magnetized.

The present invention uses granular or powdered mineral substance, vitamin substance, or ceramic as the medium which is combined with either negative hydrogen ion (H−) or hydrogen ion, where human beings and animals can take them in a capsule, tablet or powdered form according to their intended purpose. For application to food, the powdered form or tablets can be preferably used to be mixed into the food. For application to cosmetics, the liquefied or powdered form can be used to be mixed into other cosmetic products. In addition, mineral ceramic having a function of generating the negative hydrogen ion reduces the chlorine as a filter element of a water purifier, thereby enabling to produce reduced water with negative hydrogen ion (H−) which is used as safe drinking water.

### BRIEF DESCRIPTION OF THE DRAWING

FIG. 1 is a schematic cross sectional view showing an example of structure of the apparatus for producing the medium that includes the negative hydrogen ion in accordance with the present invention.

### DETAILED DESCRIPTION OF THE INVENTION

The present invention will be described in more detail. The method and apparatus of the present invention produces various substances (medium) that include negative hydrogen icons in a manner that the negative hydrogen icons stay in the substances. The method and apparatus combines and includes the negative hydrogen ion (H−) and hydrogen ion with the medium which is, for example, mineral substance, vitamin substance, mineral ceramic, etc.

The negative hydrogen ion is known to ionize as a hydride (H−) when, generally, alkali metal, alkaline earth metal, or element hydride showing metallic property at the 13th and 14th group elements are ionized. Thus, the medium including the negative hydrogen ion (H−) enters into the human body and reacts with moister therein to generate negative hydrogen ion (H−). As a consequence, harm to human cells due to the radical oxygen entering the body through the respiratory organ or those that already exist within the body or are generated from the body can be neutralized and stopped by the negative hydrogen ion or hydrogen ion. Accordingly, smooth metabolism of human cells is enhanced to improve and activate the body condition.

Further, when taking vitamin pills sold in the market, it is known that not all of the vitamin substance will be absorbed by the human body and released externally therefrom. It is also known that the vitamin substance that remained in the intestine will oxidize and harm the body. By including the hydrogen ion in the vitamin substance, the oxidized vitamin substance will be reduced, and thus, will reduce the harm to the human body. Also, mineral ceramic that can generate the negative hydrogen ion reacts with the water to generate the negative hydrogen ion. Applying this function as a filter for the water purifier, it is possible to produce reduced drinking water.

Since the present invention combines and includes the negative hydrogen ion (H−) and hydrogen ion with the medium, which is the mineral substance, vitamin substance, or mineral ceramic, it reacts with the moisture in the human body as noted above. Thus, the human body rapidly absorbs the negative hydrogen ion (H−) and hydrogen ion in the medium, where it neutralizes about 2% of the active (radical) oxygen taken from the respiratory organ as well as those that already exist within the body or generated by the body, thereby improving environment for human cells.

Living beings are all constructed from cells. Humans in their twenties are constructed from 60 trillion cells, those in their sixties are constructed from 45 trillion cells, thus, the number of cells continue to decrease with increase of age. Further, the water content of the weight ratio between a child and an elderly decreases from about 95% to 50%. However, since the water content within each cell is consistent, by taking negative hydrogen ion (H−) and hydrogen ion into the human cells, the oxidation-reduction potential of the water in the body can be lowered to promote cell activation.

The negative hydrogen ion (H−) of the present invention can be relatively easily ionized as a hydride (H−) when the alkali metal, alkaline earth metal, or the element hydride showing metallic property at the 13th and 14th group elements are ionized. Typically, coral calcium, metallic magnesium, silicon, and granite are proposed as those capable of being used at low cost for the ionization, and they are easily and safely consumed with respect to the manufacturing method and the manufactured product as described in the appended claims.

By using the magnetic mineral ceramic that is able to generate the negative hydrogen ion of the present invention as a filter for the water purifier, the water will change into negative hydrogen ion water carrying a moderate amount of reduced force. The negative hydrogen ion water produced in such a manner plays the role of eliminating the radical oxygen in the human body as well as providing nutrition to the human body because of small amount of mineral content therein. In addition, magnetic energy in the ceramic promotes the cell activation.

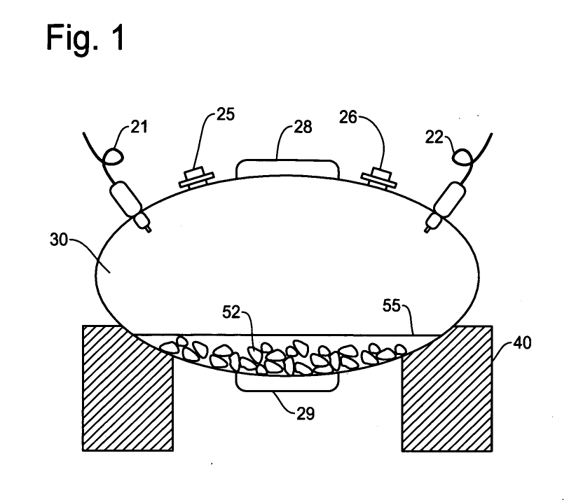

FIG. 1 is a schematic cross sectional view showing an example of structure of the apparatus for producing the medium that include the negative hydrogen ion in accordance with the present invention. In this example, the production apparatus is formed of a container 30 mounted on a base 40 where the container 30 is made of hard and strong material. In the container 30, there are medium 52 which is for example, mineral substance, vitamin substance, or magnetic mineral ceramic, and liquid hydrogen 55.

High voltage plugs 21 and 22 are attached to the container 30 for supplying high electric voltage to the inside of the container 30. Caps (or valves) 28 and 29 are also provided to the container 30 for inlet and outlet of the medium where the upper cap 28 may be used for introducing the medium into the container 30 and the lower cap 29 may be used to retrieve the treated medium 52 and the liquid hydrogen 55. Caps (or valves) 25 and 26 are provided to the container 30 for inlet and outlet of the liquid hydrogen or hydrogen gas. The caps 25 and 26 are also used for raising the inner pressure of the container 30, heating the inside of the container 30, or creating a vacuum atmosphere within the container 30.

In operation, an optional mineral substance or vitamin substance as the medium 52 in granular or powdered form is supplied to the inside of the container 30 and filled with liquid hydrogen 55 or hydrogen gas. Then after leaving them in the container 30 for a predetermined time such as about 30 minutes to 3 hours, the medium 52 is removed to be air dried, thereby producing the medium 52 which is combined and included with negative hydrogen ion.

Alternatively, after leaving the medium and the liquid hydrogen for the predetermined time (about 30 minutes to 3 hours), the liquid hydrogen is removed. Then, the medium 52 is heated at a predetermined temperature such as about 40-80° C., thereby producing the medium 52 which is combined and included with negative hydrogen ion.

Alternatively, after leaving the medium and the liquid hydrogen for the predetermined time (about 30 minutes to 3 hours), the liquid hydrogen is removed. Then, the medium 52 is pressurized at a predetermined pressure level such as about 5-1000 atm) to produce a plasma state so that hydrogen atom in the medium is converted to the negative hydrogen ion (H−), thereby producing the medium 52 which is combined and included with negative hydrogen ion.

Alternatively, after leaving the medium and the liquid hydrogen for the predetermined time (about 30 minutes to 3 hours), the liquid hydrogen or hydrogen gas is removed. Then, the medium 52 is heated at a predetermined temperature such as about 500-1,000° C. for a predetermined time length such as 1-4 hours to produce a plasma state so that hydrogen atom in the medium is converted to the negative hydrogen ion (H−), thereby producing the medium 52 which is combined and included with negative hydrogen ion.

Alternatively, after leaving the medium and the liquid hydrogen for the predetermined time (about 30 minutes to 3 hours), the liquid hydrogen or hydrogen gas is removed. The air inside of the container 30 is also removed to create an absolute or substantial vacuum state in the container 30. Then, the medium 52 is exposed to a high voltage such as about 10,000-100,000 volts for a predetermined time length such as 10-60 minutes to produce a plasma state so that hydrogen atom in the medium is excited and converted to the negative hydrogen ion (H−), thereby producing the medium 52 which is combined and included with negative hydrogen ion.

Alternatively, after leaving the medium and the liquid hydrogen for the predetermined time (about 30 minutes to 3 hours), the liquid hydrogen is removed. The hydrogen gas remains in the container 30. Then, the medium 52 is exposed to a high voltage such as about 10,000-100,000 volts for a predetermined time length such as 10-60 minutes to produce a plasma state so that hydrogen atom in the medium is excited and converted to the negative hydrogen ion (H−), thereby producing the medium 52 which is combined and included with negative hydrogen ion.

In the manufacturing method, the mineral substance or vitamin substance including the negative hydrogen ion (H−) and hydrogen ion through the treatment in the manufacturing method noted above is formed into capsules, tablets, powder, granules, in tubes or gel for medical purposes and supplemental purposes to be taken by humans, animals, and plants.

In the further aspect, powdered mineral inclusion substance and iron, calcium, magnesium, silicon and organic substance are mixed at a predetermined mixture ratio. The mixed substances are sintered to form ceramic in a porous manner with a desired size. The ceramic is supplied to the inside of the container 30 and filled with liquid hydrogen 55 or hydrogen gas. Then after leaving them in the container 30 for a predetermined time such as about 30 minutes to 3 hours, the liquid hydrogen or hydrogen gas is removed. The air inside of the container 30 is also removed to create an absolute or substantial vacuum state in the container 30. Then, the ceramic is exposed to a high voltage such as about 10,000-100,000 volts for a predetermined time length such as 10-60 minutes to produce a plasma state so that hydrogen atom in the ceramic is excited and converted to the negative hydrogen ion (H−), thereby producing the ceramic which is combined and included with negative hydrogen ion. Then, the ceramic is removed from the container 30 and magnetized.

In order to easily take in the mineral substance and vitamin substance combined and included with the negative hydrogen ion (H−) and hydrogen ion of the present invention, it should be formed into capsules, tablets, granules, or powder, etc. In order to add the mineral substance and vitamin substance combined and included with the negative hydrogen ion (H−) and hydrogen ion to food, it should be formed into granules and powder.

In order to use the magnetic mineral ceramic that can generate the negative hydrogen ion (H−) as a filter element of a water purifier, it should be formed into small spheres so that it can have a large amount of contact area with the water. The negative hydrogen ion generating magnetic mineral ceramic of the present invention should be put into a container with a large number of holes so that it can be put inside a canteen, bottle, thermos, etc.

By immersing the mineral substance and vitamin substance of the present invention in the liquid hydrogen and/or hydrogen gas, the aggregate of the molecules can be made smaller, and the individual substance can be easily absorbed into the organism of human body as well as make most of their individual characteristics.

Since the substance produced by the manufacturing method of the present invention combines and includes the negative hydrogen ion (H−) and hydrogen ion in the medium, the products using this medium will not easily oxidize, thus extends the life span of the products.

The mineral substance and vitamin substance produced by the manufacturing method of the present invention are taken into the human body and reacts with the moisture within the body to generate negative hydrogen ion (H−) and hydrogen ion, where the active (radical) oxygen are changed to good-natured oxygen, thereby improving the organic condition of the human body as well as lessening the harm to the cells by the active oxygen.

By eating the food that contains the negative hydrogen ion (H−) and hydrogen ion, it is expected that the radical oxygen is eliminated from the body. The food that includes the negative hydrogen ion (H−) and hydrogen ion no longer needs to use preservatives or can significantly reduce the amount of preservatives.

By using the magnetic mineral ceramic which can generate the negative hydrogen ion made through the manufacturing method of the present invention as a filter of the water purifier, water with a high reduction force can be obtained.

The product manufactured by the manufacturing method of the present invention does not have any harmful effects on organisms of the human body resulting from the negative hydrogen ion (H−) and hydrogen ion, which are characteristics aside from those carried by the individual mineral substance and vitamin substance.

The individual mineral substance and vitamin substance in the products manufactured by the manufacturing method of the present invention can be applied for either a medical purpose or a supplemental purpose. It is considered that such products can prevent the side effects of cancer as well as stop the damage to genes that may cause cancer. Further, by lowering the oxidation-reduction potential within the organism of the human body, the products can improve lifestyle-related diseases.

By adding granular or powdered substance containing the negative hydrogen ion (H−) and hydrogen ion manufactured by the manufacturing method of the present invention to food, it is expected that deterioration of the food is delayed, life time of the food is extended, and preservative for the food can be reduced.

By changing the water for preserving the food to the water with the negative hydrogen ion (H−) and hydrogen ion, it is expected that deterioration of the food is delayed, life time of the food is extended, and preservative for the food can be reduced.

What is claimed is:

1. A method for producing medium containing negative hydrogen ion, comprising the steps of:
an optional mineral substance or vitamin substance as a medium in granular or powdered form is supplied to an inside of a container;
the container is filled with liquid hydrogen or hydrogen gas;
leaving the medium and the liquid hydrogen or hydrogen gas in the container for a predetermined time; and
the medium is removed to be air dried, thereby producing the medium which is combined and included with negative hydrogen ion.

2. The method for producing medium as defined in claim 1, wherein after leaving the medium and the liquid hydrogen or hydrogen gas for the predetermined time, the liquid hydrogen is removed, and the medium is heated at a predetermined temperature, thereby producing the medium which is combined and included with negative hydrogen ion.

3. The method for producing medium as defined in claim 1, wherein after leaving the medium and the liquid hydrogen or hydrogen gas for the predetermined time, the liquid hydrogen is removed, and the medium is pressurized at a predetermined pressure level to produce a plasma state so that hydrogen atom in the medium is converted to the negative hydrogen ion (H−), thereby producing the medium which is combined and included with negative hydrogen ion.

4. The method for producing medium as defined in claim 1, wherein after leaving the medium and the liquid hydrogen or hydrogen gas for the predetermined time, the liquid hydrogen or the hydrogen gas is removed, and the medium is heated at a predetermined temperature for a predetermined time length to produce a plasma state so that hydrogen atom in the medium is converted to the negative hydrogen ion (H−), thereby producing the medium which is combined and included with negative hydrogen ion.

5. The method for producing medium as defined in claim 1, wherein after leaving the medium and the liquid hydrogen or hydrogen gas for the predetermined time, the liquid hydrogen or the hydrogen gas is removed, and air inside of the container is also removed to create an absolute or substantial vacuum state in the container, and the medium is exposed to a high voltage for a predetermined time length to produce a plasma state so that hydrogen atom in the medium is excited and converted to the negative hydrogen ion (H−), thereby producing the medium which is combined and included with negative hydrogen ion.

6. The method for producing medium as defined in claim 1, wherein after leaving the medium and the liquid hydrogen or hydrogen gas for the predetermined time, the liquid hydrogen is removed where the hydrogen gas remains in the container, and the medium is exposed to a high voltage for a predetermined time length to produce a plasma state so that hydrogen atom in the medium is excited and converted to the negative hydrogen ion (H−), thereby producing the medium which is combined and included with negative hydrogen ion.

7. The method for producing medium as defined in claim 1, wherein the mineral substance or vitamin substance including the negative hydrogen ion (H−) and hydrogen ion through the treatment in the manufacturing method noted above is formed into capsules, tablets, powder, granules, in tubes or gel for medical purposes and supplemental purposes to be taken by humans, animals, and plants.

8. A method for producing magnetic mineral ceramic containing negative hydrogen ion, comprising the steps of:
powdered mineral inclusion substance and iron, calcium, magnesium, silicon and organic substance are mixed at a predetermined mixture ratio;
the mixed substances are sintered to form ceramic in a porous manner with a desired size;
the ceramic is supplied to the inside of the container and filled with liquid hydrogen or hydrogen gas;
leaving the ceramic and the liquid hydrogen or hydrogen gas in the container for a predetermined time; the liquid hydrogen or hydrogen gas is removed;
air inside of the container is also removed to create an absolute or substantial vacuum state in the container;
the ceramic is exposed to a high voltage for a predetermined time length to produce a plasma state so that hydrogen atom in the ceramic is excited and converted to the negative hydrogen ion (H−), thereby producing the ceramic which is combined and included with negative hydrogen ion; and
the ceramic is removed from the container and magnetized.
